(19) United States
(12) Patent Application Publication
(10) Pub. No.: US 2022/0130522 A1
(43) Pub. Date: Apr. 28, 2022

(54) DOSE DATA MANAGEMENT SYSTEM AND METHOD

(71) Applicant: Siemens Healthcare GmbH, Erlangen (DE)

(72) Inventor: Karlheinz DORN, Kalchreuth (DE)

(73) Assignee: Siemens Healthcare GmbH, Erlangen (DE)

(21) Appl. No.: 17/508,160

(22) Filed: Oct. 22, 2021

(30) Foreign Application Priority Data
Oct. 27, 2020 (DE) 10 2020 213 497.9

(51) Int. Cl. G16H 30/20; G16H 40/67; G16H 15/00; G16H 10/60; G16H 40/20; G16H 70/20; G16H 20/10; H04L 29/06; A61B 6/00

(52) U.S. Cl. CPC G16H 30/20; G16H 40/67; G16H 15/00; G16H 10/60; A61B 6/56; G16H 70/20; G16H 20/10; H04L 63/08; G16H 40/20

(57) ABSTRACT

A method and system are for providing dose data from a cloud-based dose management system in a local Intranet. The method includes receiving study data according to at least one selection criterion from an image archiving and communication system, within the local Intranet; uploading first dose data, including or indicating at least one respective unique study-identifying characteristic value, based upon the study data received, into a cloud-based dose management system; checking whether a trigger condition is present; downloading via the dose data provisioning module, upon the checking indicating a presence of the trigger condition, second dose data, including or indicating a respective unique study-identifying characteristic value, based upon the first dose data from the cloud-based dose management system; and providing the second dose data to a reporting system or another local apparatus within the local Intranet, either automatically or upon a provisioning condition being fulfilled.

### PRIORITY STATEMENT

The present application hereby claims priority under 35 U.S.C. § 119 to German patent application number DE 102020213497.9 filed Oct. 27, 2020, the entire contents of which are hereby incorporated herein by reference.

### FIELD

Example embodiments of the invention generally relate to a method for providing dose data from a cloud-based dose management system in a local Intranet and to a corresponding system.

### BACKGROUND

Medical institutions are increasingly using central, cloud-based dose management systems. In this way local infrastructure can be reduced or avoided, as a result of which maintenance costs are reduced and data backups can often be carried out in a simple and cost-effective manner. At the same time, such solutions present new challenges: for instance it is further desirable in the case of hospitals, for instance, for applied dose values to be able to be inserted into locally produced patient study reports.

Applied dose values are understood here to mean values relating to the radiation doses to which patients are exposed during the course of what are known as “studies” (or: “exams”). For instance, one such study can consist of a series of computed tomography scans, each of which can represent a (radiation) dose-relevant event, known as a “dose event”. Further dose events can be for instance the administration of a contrast agent or even the ingestion or absorption of a radioactive material by a patient (for instance in the case of poisoning or for a medical examination).

### SUMMARY

The inventors have discovered that a further disadvantage of existing systems resides in physicians or specialist personnel usually creating a report after a study has been carried out. This is typically dictated with the aid of what is known as a reporting system and should contain the relevant dose data. It is laborious for the dictating person to open or process further software applications during the dictation, which is frequently carried out with dictation software, in order to be able to examine the dose values so that it is then possible to insert them into the report.

Furthermore, the inventors have discovered that medical institutions are under increasing pressure from national agencies to provide dose data (or: dose information) based upon internationally comparable standards, in order to be able to compare the applied dose values with defined reference values.

The inventors have discovered that another problem is that reference values and the dose data can in most cases not be readily compared, since to some extent studies carried out in various institutions differ considerably in terms of their content and cannot therefore be compared directly with one another. The use of different apparatuses (e.g. device or software solutions), different evaluation and management software, different national specifications etc. additionally hampers the comparability.

Furthermore, the inventors have discovered that a simple method is needed in order to transmit dose values into a higher-level dose registry, for instance a national dose registry, which is frequently required within the scope of financial support. Since systems and software solutions other than those used in the individual medical institutions themselves are in turn typically used for this purpose, it was up to now a complicated undertaking. Conversely, there is also the need to be able to provide reference values from such national dose registries easily in a medical institution and to be able to analyze the same in as synchronized a manner as possible with other dose registries, for instance global dose registries.

According to a first embodiment, the invention provides a method for providing dose data from a cloud-based dose management system, CBDMS, in a local Intranet, LIN, wherein the method comprises at least:

receiving, at a medicine data gateway, MDGW, within the local Intranet, study data according to at least one predefined selection criterion of an image archiving and communication system, PACS, within the local Intranet;

uploading, via the medicine data gateway, MDGW, at least first dose data based upon the received study data, into a cloud-based dose management system, CBDMS, wherein the first dose data comprises or indicates at least one respective unique study-identifying characteristic value, ESIK;

checking, via a dose data provisioning module, DDBM, which is executed in the medicine data gateway, MDGW, within the local Intranet, whether a predefined trigger condition is present, and if this is the case:

downloading, via the dose data provisioning module, DDBM, second dose data based upon the first dose data from the cloud-based dose management system, CBDMS, wherein the second dose data comprises or indicates the respective unique study-identifying characteristic value, ESIK; and

providing the second dose data to a reporting system, REP, or another local apparatus within the local Intranet, either automatically or if a predefined provisioning condition is fulfilled.

According to a second embodiment, the invention provides a system for providing dose data from a cloud-based dose management system, CBDMS, comprising:

a medicine data gateway, MDGW, within a local Intranet of the system, wherein the medicine data gateway, MDGW, is embodied to execute a dose data provisioning module, DDBM;

a reporting system, REP, within the local Intranet; and

a cloud-based dose management system, CBDMS, outside of the local Intranet;

wherein the system is configured to execute the method according to one of the embodiments of the first aspect of the invention.

According to a third embodiment, the invention provides a computer program product, which comprises executable program code, which is embodied, when it is executed, to execute the method according to one of the embodiments of the invention.

According to a fourth embodiment, the invention provides a computer-readable, non-volatile data storage medium, which comprises executable program code, which is embodied, when it is executed, to execute the method according to one of the embodiments of the invention.

According to a fifth embodiment, the invention provides a data stream, which comprises executable program code, or is designed to provided executable program code, wherein the executable program code is embodied, when it is executed, to execute the method according to one of the embodiments of the invention.

According to an embodiment, the invention provides a method for providing dose data from a cloud-based dose management system in a local Intranet, comprising: receiving, at a medicine data gateway within the local Intranet, study data according to at least one selection criterion from an image archiving and communication system, within the local Intranet;

uploading, via the medicine data gateway, at least first dose data based upon the study data received, into a cloud-based dose management system, the first dose data including or indicating at least one respective unique study-identifying characteristic value;

checking, via a dose data provisioning module executed in the medicine data gateway within the local Intranet, whether a trigger condition is present;

downloading via the dose data provisioning module, upon the checking indicating a presence of the trigger condition, second dose data based upon the first dose data from the cloud-based dose management system, the second dose data including or indicating a respective unique study-identifying characteristic value; and

providing the second dose data to a reporting system or another local apparatus within the local Intranet, either automatically or upon a provisioning condition being fulfilled.

According to an embodiment, the invention provides a system for providing dose data from a cloud-based dose management system, comprising:

a medicine data gateway, within a local Intranet of the system, the medicine data gateway being embodied to execute a dose data provisioning module;

a reporting system, within the local Intranet; and

a cloud-based dose management system, outside of the local Intranet;

the system being configured to
receive, at a medicine data gateway within the local Intranet, study data according to at least one selection criterion from an image archiving and communication system, within the local Intranet;
upload, via the medicine data gateway, at least first dose data based upon the study data received, into a cloud-based dose management system, the first dose data including or indicating at least one respective unique study-identifying characteristic value;
check, via a dose data provisioning module executed in the medicine data gateway within the local Intranet, whether a trigger condition is present;
download via the dose data provisioning module, upon the checking indicating a presence of the trigger condition, second dose data based upon the first dose data from the cloud-based dose management system, the second dose data including or indicating a respective unique study-identifying characteristic value; and
provide the second dose data to a reporting system or another local apparatus within the local Intranet, either automatically or upon a provisioning condition being fulfilled.

According to an embodiment, the invention provides a non-transitory computer program product storing executable program code, embodied, when executed, to execute the method of an embodiment.

According to an embodiment, the invention provides a non-transitory computer-readable, non-volatile data storage medium storing executable program code embodied, when executed, to execute the method of an embodiment.

### BRIEF DESCRIPTION OF THE FIGURES

The invention and the technical field are explained in more detail below based upon the figures. Reference is made to the invention not being restricted by the example embodiments shown. In particular, it is, unless explicitly shown otherwise, also possible to extract partial aspects of the circumstances explained in the figures and to combine them with other component parts and knowledge from the present description and/or figures. In particular, it is noted that the figures and in particular the scales shown are only schematic. The same reference characters refer to the same objects so that explanations from other figures can possibly be used in addition.

The numbering of method steps is used primarily to distinguish between them and is not necessarily intended to imply a special sequence if this is not described clearly or explicitly from the content or context.

In the figures:

FIG. 1 shows a schematic representation to explain a method in accordance with an embodiment according to the first aspect of the present invention and to explain a system in accordance with an embodiment according to the second aspect of the present invention;

FIG. 2 shows a schematic flowchart for the method according to FIG. 1;

FIG. 3 shows a schematic representation to explain a method in accordance with a further embodiment according to the first aspect of the present invention and to explain a system in accordance with a further embodiment according to the second aspect of the present invention;

FIG. 4 shows a schematic flowchart for the method according to FIG. 3;

FIG. 5 shows a schematic representation to explain a method in accordance with yet another embodiment according to the first aspect of the present invention and to explain a system in accordance with yet another embodiment according to the second aspect of the present invention;

FIG. 6 shows a schematic flowchart for the method according to FIG. 5;

FIG. 7 shows a schematic block diagram of a computer program product in accordance with an embodiment according to the third aspect of the present invention; and

FIG. 8 shows a schematic block diagram of a data storage medium in accordance with an embodiment according to the fourth aspect of the present invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (procesor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

According to a first embodiment, the invention provides a method for providing dose data from a cloud-based dose management system, CBDMS, in a local Intranet, LIN, wherein the method comprises at least:

receiving, at a medicine data gateway, MDGW, within the local Intranet, study data according to at least one predefined selection criterion of an image archiving and communication system, PACS, within the local Intranet;

uploading, via the medicine data gateway, MDGW, at least first dose data based upon the received study data, into a cloud-based dose management system, CBDMS, wherein the first dose data comprises or indicates at least one respective unique study-identifying characteristic value, ESIK;

checking, via a dose data provisioning module, DDBM, which is executed in the medicine data gateway, MDGW, within the local Intranet, whether a predefined trigger condition is present, and if this is the case:

downloading, via the dose data provisioning module, DDBM, second dose data based upon the first dose data from the cloud-based dose management system, CBDMS, wherein the second dose data comprises or indicates the respective unique study-identifying characteristic value, ESIK; and

providing the second dose data to a reporting system, REP, or another local apparatus within the local Intranet, either automatically or if a predefined provisioning condition is fulfilled.

The medicine data gateway, MDGW, can be the “Teamplay” or “teamplay” (registered trademark) receiver by the company Siemens Healthineers or a similar product, for instance.

The cloud-based dose management system, CBDMS, can have further functions or be provided as part of a more extensive cloud functionality, in which studies, benchmarks, boxplots and/or suchlike can be stored, for instance, in particular in de-identified form owing to considerations of data protection.

The cloud-based dose management system, CBDMS, can advantageously provide in particular the functionality of a global dose registry. Contrary to national dose registries, this global dose registry can provide dose data which is globally binding, such as e.g. benchmarks, reference values inter alia, and on/in a wide variety of modalities, software applications, medical institutions and suchlike.

The study data can be present in particular in the form of structured reports, SRs, according to the DICOM standard. “DICOM” is a registered trademark and stands for “Digital Imaging and Communications in Medicine”. Whenever the DICOM standard is mentioned herein, it can be understood to mean in particular the current version of the DICOM standard PS3.1 2020d, wherein structured reports can also be used according to a preceding or a subsequent DICOM standard version. Within the scope of the first dose data, complete studies can in particular be uploaded into the cloud-based dose management system, CBDMS.

The dose data provisioning module, DDBM, can, as is explained in more detail again below, comprise a plurality of mechanisms. In particular, a push function and/or a pull function can be provided. Moreover, the method is suited to supplying global or national dose databases or dose registries with data and/or to always receiving current dose reference values therefrom. These dose reference values can then be provided linked with performed studies (exams) in order to present physicians or other evaluators of studies with a comprehensive image.

The reporting system can be, for instance, the “Powerscribe” (registered trademark) system by the company Nuance or a comparable system.

A local apparatus is in particular a device which is available in the local Intranet or a software application which is available there or a local computing facility, which executes such a software application. For instance, the reporting system or a radiology information system can be understood to be a local apparatus. A personal computer (PC) of a physician can also represent a local apparatus, as well as management software which is or can be executed on such a PC.

Embodiments of the invention described here with all its associated ideas and principles is described based upon the specific problems of dose management. It is apparent, however, that an analog, i.e. identical, application to similar, closely related problems is possible, for instance to contrast management or protocol management.

Accordingly, mention could thus be given to:
“contrast data” or “protocol data” instead of “dose data”;
a “contrast data provisioning module CDBM” or a “protocol data provisioning module PDBM” in each case instead of the “dose data provisioning module DDBM”;
in general “contrast management” or “protocol management” instead of “dose data management”;
and so on.

If a cloud-based contrast management system and/or a cloud-based protocol management system are therefore provided instead of (or in addition to) the cloud-based dose management system, a global contrast registry and/or a global protocol registry can therefore also be provided accordingly.

Further embodiments, features, advantages, variants and development possibilities result from the subclaims and from the description with reference to the figures.

According to a few preferred embodiments, variants or developments of embodiments, the unique study-identifying characteristic value, ESIK, is at least one of the following characteristic values and/or is based on at least one of the following characteristic values:
an accession number, AN, which represents a reference number in a radiology information system within the local Intranet, LIN;
a patient identification characteristic value, PIK; and/or
a StudyInstanceUID, which is designed to uniquely identify a study associated with a structured report, SDR.

With a respective unique study-identifying characteristic value, ESIK (i.e. available for each study), which is used to some extent as a “magic cookie”, a data exchange can therefore be enabled between devices and services of different manufacturers, different hospitals, different software providers and suchlike.

The accession number is referred to in the DICOM standard by the element number (0008,0050). It links a process established in the radiology information system, RIS, with the images resulting from this process, which are stored in the image archive. The accession number is awarded by the RIS and stored explicitly in the generated or imported DICOM objects, so that when images are viewed from an image archive a user is able to reproduce the process in the RIS to which the viewed images can be assigned.

According to a few preferred embodiments, variants or developments of embodiments, the dose data provisioning module, DDBM, comprises a reactive pull module, RPM (or consists of such a reactive pull module, RPM), which
provides a URL, which can be called by the local apparatus within the local Intranet, in order to request the second dose data via the local apparatus; wherein the URL comprises at least one respective unique study-identifying characteristic value, ESIK;
wherein the predefined trigger condition comprises the receiving of a call of the provided URL; and
wherein the second dose data is provided by the reactive pull module, RPM, of the local apparatus.

Therefore such URLS, with which required dose data in the pull method is easily provided upon request, can be provided to any local apparatuses, such as e.g. radiology information systems, reporting systems and suchlike. In this way there is conveniently no need for a user of these local apparatuses to still simultaneously separately manage a dose management system in addition to using this apparatus.

According to a few preferred embodiments, variants or developments of embodiments, the reactive pull module, RPM, provides the local apparatus with a service access token, SAT, which is checked in the course of the URL call on the local apparatus via the reactive pull module, RPM, in order to authenticate the call, wherein the predefined trigger condition comprises a successful authentication of the service access token, SAT.

According to a few preferred embodiments, variants or developments of embodiments, the local apparatus is a radiology information system, RIS.

According to a few preferred embodiments, variants or developments of embodiments, the dose data provisioning module, DDBM, comprises a proactive push module, PPM, or consists of such a proactive push module, PPM,

wherein the predefined trigger condition comprises the receiving of an item of information from the cloud-based dose management system, CBDMS, that a study has been fully uploaded into the cloud-based dose management system, CBDMS. In this way, within the scope of the second dose data, dose values can be “pushed” into the reporting system, for instance, so that they are automatically available to a physician or other specialist personnel when a report is created (e.g. dictated).

According to a few preferred embodiments, variants or developments of embodiments, the medicine data gateway, MDGW, de-identifies the first dose data and uploads this in de-identified form into the cloud-based dose management system, CBDMS,

wherein the medicine data gateway, MDGW, in particular via the dose data provisioning module, DDBM, re-identifies the downloaded second dose data based upon the respective, unique study-identifying characteristic value, ESIK. De-identification is to be understood to mean in particular that de-identified data or characteristic values (without additional data) cannot be associated with a specific patient, whereas re-identified data can be associated with a specific patient.

According to a few preferred embodiments, variants or developments of embodiments, the predefined provisioning condition, VDB, comprises a positive result of a check to determine whether an incomplete order currently exists on the reporting system and is linked to a unique study-identifying characteristic value, ESIK, which is associated with the second dose data.

According to a few preferred embodiments, variants or developments of embodiments, information about a standard protocol assignment and/or standard reference values is provided as part of the second dose data or in addition to the second dose data.

The second dose data can comprise, for instance, desired dose parameters, e.g. CTDIVOL and/or DLP, for instance for an entire study (also referred as “exam”) or just for an individual dose event. The second dose data can be transferred in various formats, for instance in one (or more) of the following formats:

text format (.txt file)

PDF format (.pdf file)

Json format

DICOM format (as a structured report dose RDSR).

Advantageously information about a standard protocol mapping and/or standard reference values can be provided as part of the second dose data, or in addition to the second dose data, to the local apparatus. For instance, the cited dose parameters can be supplemented with the standard mappings and standardized protocols such as RadLex or LOINC. Alternatively or in addition, the dose events can themselves also be attached and/or an associated dose reference value, which is automatically determined as a function of the standardized protocol assignment.

RadLex is a LEXicon of RADiological information, which was produced by the “Radiological Society of North America”, RSNA. It represents an ontology, the aim of which is to develop useful vocabulary for radiologists.

LOINC (“Logical Observation Identifiers Names and Codes”) is an international, substantially English-speaking system for unique encryption of medical examinations, in particular laboratory examinations, which is published by the Regenstrief Institute (USA). LOINC can therefore also be referred to as a superset to RadLex, or overlaps exist between RadLex and LOINC and thus LOINC-compatible RadLex protocols.

According to a few preferred embodiments, variants or developments of embodiments, the dose data provisioning module, DDBM, comprises a dose registration module, DRM, or consists of such a dose registration module, DRM, wherein the cloud-based dose management system, CBDMS, if a study has been uploaded entirely into the cloud-based dose management system, CBDMS, informs a dose registration module, DRM, executed in the medicine data gateway, MDGW, thereof;

whereupon the dose registration module, DRM, provides the second dose data to a registry site service system, REG, as the local apparatus within the local Intranet;

wherein the registry site service system, REG, transmits third dose data based upon the second dose data to a national dose registry computing facility, NDRR.

According to a few preferred embodiments, variants or developments of embodiments, the registry site service system, REG, receives (preferably regularly) dose benchmark data from the national dose registry computing facility, NDRR, whereupon the registry site service system, REG, informs the dose registration module, DRM, about the availability of new dose benchmark data, whereupon the dose registration module, DRM, uploads the new dose benchmark data into the cloud-based dose management system, CBDMS.

According to a few preferred embodiments, variants or developments of embodiments, the cloud-based dose management system, CBDMS, additionally functions as a global dose registry, as a cloud-based contrast management system, CBKMS, and/or as a cloud-based protocol management system, CBPMS.

A contrast management system is to be understood to mean, for instance, a software (and/or hardware) package, which is configured to receive, read out, store, share, compare injection programs for contrast agent for instance from the PACS, the radiology information system, RIS, and/or from an injection system, and/or to represent data (in particular contrast data) graphically.

A protocol management system is to be understood to mean for instance a software (and/or hardware) package, which is configured to provide multi-modal access to monitor and possibly set/change imaging protocols.

According to a second embodiment, the invention provides a system for providing dose data from a cloud-based dose management system, CBDMS, comprising:

a medicine data gateway, MDGW, within a local Intranet of the system, wherein the medicine data gateway, MDGW, is embodied to execute a dose data provisioning module, DDBM;

a reporting system, REP, within the local Intranet; and

a cloud-based dose management system, CBDMS, outside of the local Intranet;

wherein the system is configured to execute the method according to one of the embodiments of the first aspect of the invention.

According to a third embodiment, the invention provides a computer program product, which comprises executable program code, which is embodied, when it is executed, to execute the method according to one of the embodiments of the invention.

According to a fourth embodiment, the invention provides a computer-readable, non-volatile data storage medium, which comprises executable program code, which is embodied, when it is executed, to execute the method according to one of the embodiments of the invention.

According to a fifth embodiment, the invention provides a data stream, which comprises executable program code, or is designed to provided executable program code, wherein the executable program code is embodied, when it is executed, to execute the method according to one of the embodiments of the invention.

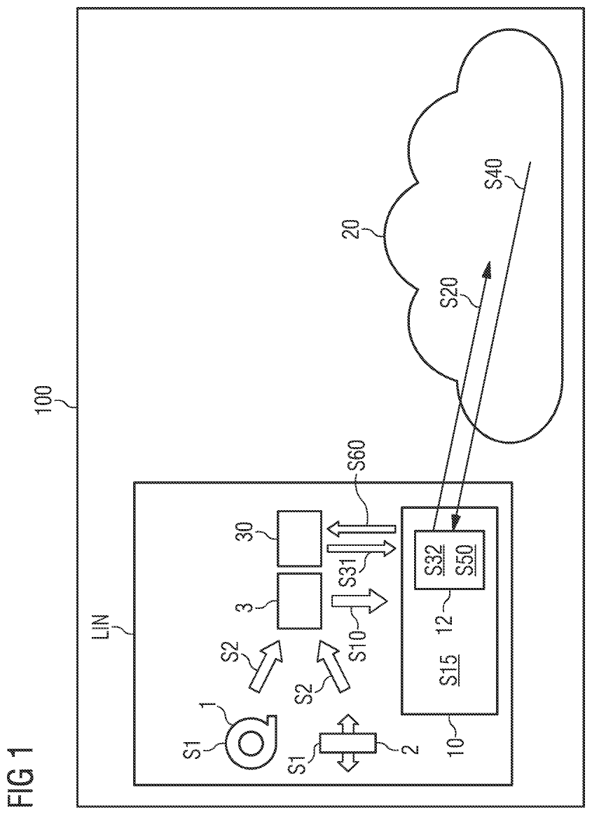

Embodiments of the invention are described in more detail below with reference to the figures. In the pairs of FIGS. 1/2,3/4 and 5/6, an embodiment of the inventive method or of the inventive system is described in more detail in each case. It is apparent that the various embodiments are independent of one another, but can also be provided in any combination with one another.

The example embodiments described with the aid of the figures relate to dose management; as was already explained in detail above, however, there can likewise be provision (alternatively or in addition) for a contrast management or a protocol management, with corresponding changes or additions to the references of the individual features, method steps and system components.

FIG. 1 shows a schematic representation to explain a method in accordance with an embodiment according to the first aspect of the present invention, i.e. a method for providing dose data from a cloud-based dose management system, CBDMS, in a local Intranet LIN. FIG. 1 moreover explains a system 100 in accordance with an embodiment according to the second aspect of the present invention, i.e. a system for providing dose data from a cloud-based dose management system, CBDMS, in a local Intranet LIN.

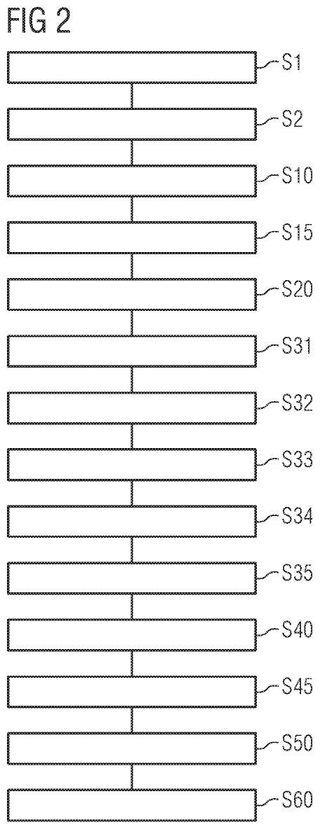

The flowchart in FIG. 2 belongs to FIG. 1, and shows individual method steps again in association therewith.

In one step S1, which is optionally part of the method, medical data in the form of structured reports SR is generated by at least one medical data source or provided in other ways, in particular structured reports SR according to the DICOM standard.

The medical data source can be a medical imaging apparatus 1, for instance, like a computed tomography system, a magnetic resonance tomography system, an ultrasound imaging apparatus or suchlike.

For instance, a structured report, SR, can be what is known as a “Dose RDSR” with SOP Class UID=1.2.840.10008.5.1.4.1.1.88.67, or what is known as a “MAMMO-CAD-SR” with SOP Class UID=1.2.840.10008.5.1.4.1.1.88.50 or what is known as a “CT Performed Procedure Protocol Storage” with SOP Class UID=1.2.840.10008.5.1.4.1.1.200.3 or suchlike. SOP Class UIDs are defined in the DICOM standard, wherein SOP stands for “service object pair”, “class” expresses a class, and “UID” stands for “unique identifier”.

The medical data source can alternatively or in addition also be an injection system 2 (injector), which provides a structured report SR in particular according to the DICOM standard. For instance, the injection system can provide what is known as a “Performed Imaging Agent Administration SR” with SOP Class UID=1.2.840.10008.5.1.4.1.1.88.75.

Further devices are also conceivable, which can provide structured reports SRs.

In a step S2, which is optionally part of the method, the structured reports SR provided by the at least one medical data source 1, 2 are provided, in particular transmitted, to an image archiving and communication system, PACS 3, within the local Intranet LIN, and received at the PACS 3, wherein PACS stands for “picture archiving and communication system”.

In a step S10, study data is received by the PACS 3 within the local Intranet LIN at a medicine data gateway, MDGW 10, within the local Intranet LIN according to at least one predefined selection criterion, in particular as a response to a query of the medicine data gateway, MDGW 10, to the PACS 3.

The medicine data gateway, MDGW 10, can, in order to mention examples of the predefined selection criterion, query all studies within a specific date range (e.g. time frame) and/or all studies with a special SOP Class UID and thereupon receive the same from the PACS 3.

In an optional step S15, the medicine data gateway, MDGW 10, can carry out the following data manipulations on the received data:
only those tags (or studies with such tags) which are assigned to a permitted list (“whitelist” or “allowlist”) are retained and/or such tags or studies which are assigned to a blocked list (“backlist” or “blocklist”) are removed;
the remaining tags are converted into another data format, in particular a generally used data format such as XML or Json.

The permitted list and/or the blocked list can be based for instance on considerations or obligations of data protection, e.g. according to the European General Data Protection Regulation (GDPR) or according to the American Health Insurance Portability and Accountability Act (HIPAA). In addition or alternatively, the permitted list and/or the blocked list can be based on considerations relating to individual features or functions of the inventive system.

In a step S20, at least first dose data based on the received study data is uploaded into a cloud-based dose management system, CBDMS 20, via the medicine data gateway, MDGW 10. The first dose data can be in particular the tags or studies remaining after the optional step S15, it can however also be extracts from these remaining studies.

The first dose data comprises or indicates at least one respective unique study-identifying characteristic value, ESIK, which can also be referred to as cookie or as “magic cookie”.

This unique study-identifying characteristic value, ESIK, can be for instance at least one of the following characteristic values and/or be based on at least one of the following characteristic values:
an accession number, AN, which represents a reference number in a radiology information system, RIS 30, within the local Intranet, LIN;
a patient identification characteristic value, PIK; and/or
a StudyInstanceUID, which is designed to uniquely identify a study associated with a structured report SR.

Owing to considerations of data protection, it is advantageous, in most countries, if sensitive data, such as for instance patient data, medical history etc., is not available in a cloud-based data memory such that it can be assigned to a specific patient.

In the optional step S15, a de-identification of the study data can therefore also advantageously be carried out before this is uploaded in step S20. The unique study-identifying characteristic value, ESIK, can therefore be replaced in the uploaded data by a de-identified, uniquely identifying characteristic value diESIK, which cannot be assigned to a specific patient, wherein the medicine data gateway, MDGW 10, within the local Intranet LIN, retains the information to which unique study-identifying characteristic value, ESIK, this diESIK corresponds. The diESIK therefore indicates a corresponding ESIK, without openly storing this information in the cloud-based system.

The uploading S20 is carried out in particular in that many different items of SOP Class UID-based DICOM study data are stored in each case in a study and institution-dependent cloud container, for instance in accordance with a hierarchy according to institution/study/SOP Class UID. With respect to the institution, a “tenant” is often mentioned, wherein this institution is generally that which sets up or maintains the local Intranet LIN.

Web applications such as for instance the “teamplay dose web application” by the company Siemens Healthineers can represent the uploaded study information in a study and/or event-dependent view, for instance, depending on the purpose for which the display is to be used.

As shown in FIG. 1, the local Intranet LIN, which can be the local Intranet of a hospital, a research institute or suchlike, can also be inter alia a radiology information system, RIS 30. This RIS 30 therefore represents a local apparatus within the local Intranet LIN.

Via a dose data provisioning module, DDBM, which, according to the present embodiment, is embodied as a reactive pull module, RPM 12, a URL (universal resource locator) has been provided to the radiology information system, RIS 30, within the scope of a web service API produced by the reactive pull module, RPM 12.

This URL can be called on or by the radiology information system, RIS 30, in order to request dose data which is subsequently to be referred to as “second dose data”. This can be identical to the first dose data, but can also be based on the first dose data and optionally comprise further data, such as is explained in more detail below.

The URL moreover comprises, when it is called, at least one respective unique study-identifying characteristic value, ESIK, which can be transferred thereto as a parameter by the radiology information system, RIS 30, irrespective of the context (e.g. with respect to a particular patient) in which a user activates the URL, for instance.

Via the reactive pull module, RPM 12, what is known as a “service access token”, SAT, may have been transferred to the radiology information system, RIS 30, for instance via an encrypted email. The SAT is also used as an additional parameter of the called URL.

In a step S31 of the method, a querying URL call is received by the radiology information system, RIS 30, on the reactive pull module, RPM 12.

In a step S32 of the method, a check is carried out to determine whether the correct SAT was added to the URL call as the parameter. If this authentication is successful, this is a trigger condition (which is adequate in the present example described) for the subsequent step S40.

A failure during the authentication in step S32 results in the method stopping in a step S33 (see FIG. 2).

In the step S40, the second dose data is downloaded based upon (or identically to) the first dose data from the cloud-based dose management system, CBDMS 20, via the reactive pull module, RPM 12. The second dose data comprises or indicates the respective unique study-identifying characteristic value, ESIK, for instance by it comprising a de-identified unique study-identifying characteristic value, diESIK.

In the in practice majority of cases whereby the method is designed so that the de-identified unique study-identifying characteristic values, diESIK, are present in the cloud-based dose management system, CBDMS 20, a check is firstly carried out in a step S34 (see FIG. 2) to determine whether at least one unique study-identifying characteristic value, ESIK, is present as a parameter of the called URL.

If this is the case, in a step S35 (see FIG. 2) a user token is produced based upon the SAT token, which is used to obtain an item of association information between the corresponding unique study-identifying characteristic value, ESIK, and the corresponding de-identified unique study-identifying characteristic value, diESIK, from the medicine data gateway, MDGW 10, so that in step S40, the correct second dose data can also be downloaded. This ensures that the query, which is transmitted from the reactive pull module, RPM 12, is transmitted to the cloud-based dose management system, CBDMS 20, is itself de-identified, i.e. does not contain any unique study-identifying characteristic value, ESIK, in plain text.

In a step S50 (see FIG. 2) the reactive pull module, RPM 12, checks to determine whether an incomplete order is currently available to a reporting system, REP 40, within the local Intranet LIN, the order being associated with a unique study-identifying characteristic value, ESIK, according to the second dose data. One such incomplete order can be an order, the processing or operation of which was not yet started, or the processing or operation of which is still in progress. As an example of the unique study-identifying characteristic value, ESIK, what is known as the accession number is in particular offered here.

A re-identification S45 (see FIG. 2) of the second dose data is in turn also possible here and required in some embodiments since in the reporting system, REP 40, the unique study-identifying characteristic value, ESIK, is typically available in plain text, while in some embodiments the second dose data downloaded in step S40 only comprises de-identified unique study-identifying characteristic values, ESIK. Different variants are apparently conceivable here, for instance a re-identification S45 in the form that each diESIK is replaced by the underlying ESIK.

If the check in step S50 is successful, in step S60 the second dose data is provided to the radiology information system, RIS 30, from which the URL was called. The second dose data can comprise for instance desired dose parameters, e.g. CTDIVOL and/or DLP, for instance for an entire study (also referred to as exam) or only for a single dose event. The second dose data can be transmitted in various formats, for instance in one (or several) of the following formats:

text format (.txt file)

PDF format (.pdf file)

Json format

DICOM format (as a structured report dose RDSR).

All of these requirements and/or variants or options can be provided in different embodiments to the URL as parameters.

It is apparent that in this method another local apparatus can appear within the local Intranet LIN instead of the radiology information system, RIS 30.

Advantageously, information about a standard protocol assignment and/or standard reference values can be provided as part of the second dose data, or in addition to the second dose data, to the local apparatus (here: radiology information system, RIS 30). For instance, the cited dose parameters can be supplemented with the standard mappings and standardized protocols such as RadLex or LOINC. Alternatively or in addition, the dose events can themselves also be added and/or an associated dose reference value, which is automatically determined as a function of the standardized protocol assignment.

In order to supplement the second dose data, the reactive pull module, RPM 12, can call an API provided by the cloud-based dose management system, CBDMS 20, in order to obtain the corresponding standard mapping for a specific study and to add the same to the second dose data. The provision S60 of the second dose data, with or without supplementation, can likewise take place in different protocols, for instance via the “DICOM send” protocol, with an http call, with a REST protocol or another type of file sharing.

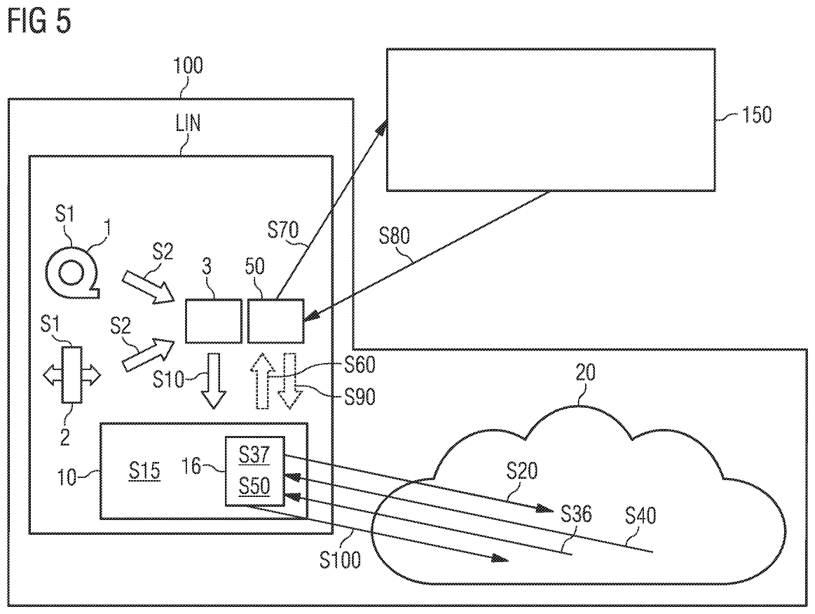

Standard reference values can be retrieved by a dose registration module, DRM, by the reactive pull module, RPM 12, wherein one such dose registration module, DRM, is explained in more detail below in conjunction with FIG. 5.

One such composition of reference values, which relate to individual dose events and the actual dose parameters (dose values) enables a completely new type of information display. In this way, the effects of different studies on a patient, which studies have in each case a number of dose events on different body parts, can for the first time be accumulated both with respect to the radiation doses actually applied and also with respect to the dose reference values.

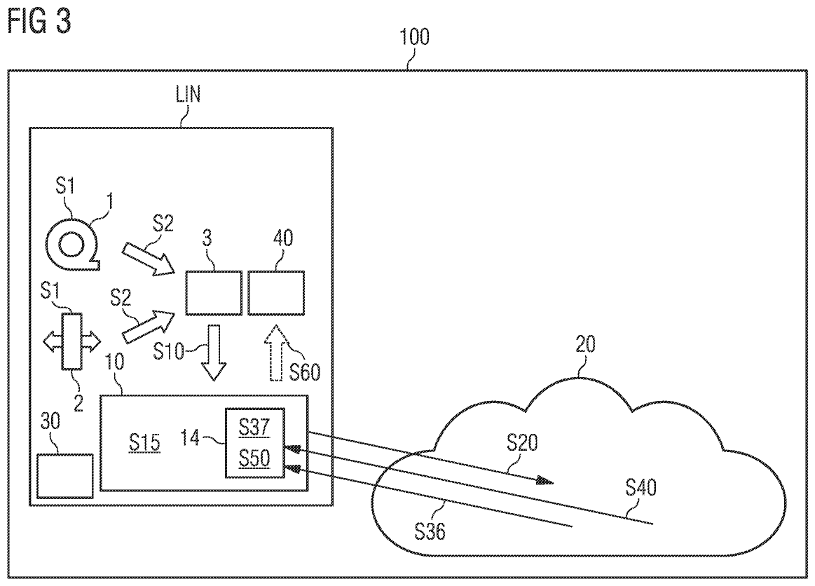

FIG. 3 shows a schematic representation to explain a method according to a further embodiment of the present invention, i.e. a method for providing dose data from a cloud-based dose management system, CBDMS, in a local Intranet LIN. FIG. 3 moreover explains a system 100 in accordance with a further embodiment according to the second aspect of the present invention, i.e. a system for providing dose data from a cloud-based dose management system, CBDMS, in a local Intranet LIN.

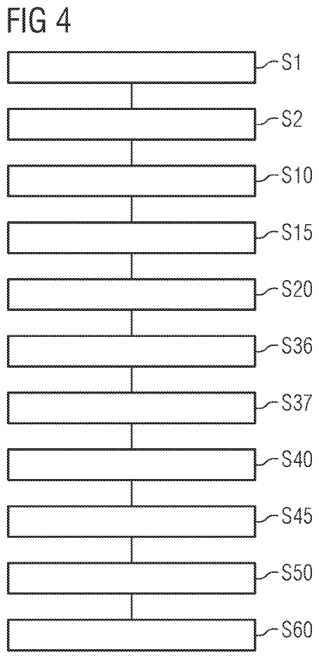

The flowchart in FIG. 4 which represents individual method steps again schematically in association therewith belongs to FIG. 3.

Contrary to the method according to FIG. 1 and FIG. 2, which has essentially described a pull mechanism, a push mechanism is currently described. The method according to FIG. 3 and FIG. 4 is extensively the same as or similar to the method according to FIGS. 1 and 2, so that only the differences therefrom are explained below. Similarly, the methods according to FIGS. 1 to 4 can also be combined, i.e. so that both a push and also a pull mechanism can be provided.

Accordingly, the case is described here in that the dose data provisioning module, DDBM, comprises a proactive push module, PPM 14 instead of the reactive pull module, RPM 12, wherein it is apparent that a dose data provisioning module, DDBM, can also comprise both the proactive push module, PPM, and also the reactive pull module, RPM 12.

In respect of FIG. 3 and FIG. 4, the steps S10 to S20 are carried out as described with the optional steps S1 and/or S2.

Also in the present case, a trigger condition is checked by the dose data provisioning module, DDBM. Contrary to the afore-described embodiment, the trigger condition is however checked as follows:

In a step S36, the cloud-based dose management system, CBDMS 20, informs the proactive push module, PPM, executed by the medicine data gateway, MDGW 10, that in step S20 a new study was uploaded entirely into the cloud-based dose management system, CBDMS 20. Optionally this can only then occur if in a special configuration software, e.g. a configuration page of a web application, this was released for specific medical data sources 1, 2.

Therefore, in a step S37, a check can be carried out by the proactive push module, PPM 14, that a corresponding item of information (or message, or notification) is present as a result of the cloud-based dose management system, CBDMS 20 and optionally a check can additionally be carried out to determine whether the push mechanism is desired for this specific study (as a function of e.g. modality, i.e. medical data source, inter alia). In the latter case, the trigger condition thus consists of two separate partial conditions, which must both be fulfilled.

If the result of the check in step S37 is positive, i.e. the trigger condition is fulfilled, steps S40 to S60 ensue as described already in detail above, wherein instead of the reactive pull module, RPM 12, the proactive push module, PPM 14, is now active.

Moreover, in this case the step S60 is modified, since with the push mechanism, no query precedes on account of a URL call.

Instead, in this embodiment, there is provision for the local apparatus, to which the second dose data is transmitted (with or without supplements) to be the reporting system, REP 40. The reporting system, REP 40, has provided a URL to the proactive push module, PPM 14, by which URL the second dose data can be transmitted. Via the URL provided by the reporting system, REP 40, it is therefore also ensured that the reporting system, REP 40, is also actually ready and able to receive the second dose data. On account of the provided URL, it is at least expected that the reporting system, REP 40, is either able to receive the second dose data (with or without supplements) or that this responds with an error if it is (currently) not able to receive it.

The correct assignment of the second dose data provided in the push method by the proactive push module, PPM 14, is in turn assured by the respective unique study-identifying characteristic value, ESIK, which functions as a “magic cookie”, with the afore-described optional variants in respect of de-identification and re-identification by the medicine data gateway, MDGW 10 (specifically: by an identification service, which is provided by the medicine data gateway, MDGW 10).

Therefore, in step S50, the URL provided by the reporting system, REP 40, is called by the proactive push module, PPM 14, in order to check the provisioning condition, i.e. to check whether an uncompleted order, which is associated with the unique study-identifying characteristic value, ESIK, is currently present in the reporting system, REP 40, such as was already explained with reference to FIG. 1 and FIG. 2. In particular, the “accession number” is suitable. The accession number can therefore be used as a “magic cookie” between otherwise completely different systems: on the one hand the reporting system, REP 40, which is installed and executed in the local Intranet, LIN i.e. typically in the secured environment of a hospital, and on the other hand the cloud-based dose management system, CBDMS 20, which is implemented as a globally used public cloud solution.

In this regard, the identification/de-identification method described mediates in addition between the requirements that in the local Intranet, LIN, data must be assigned (or at least assignable) on the one hand to the patient, whereas in a cloud solution this is unwanted.

In the step S60, with a positive provisioning condition the URL can finally be called, in order to transfer, i.e. to provide, the second dose data (with or without supplements). With respect to the second dose data (with or without supplements), the variants and options described above are in turn possible.

FIG. 5 shows a schematic representation to explain a method according to yet another embodiment of the present invention, i.e. a method for providing dose data from a cloud-based dose management system, CBDMS, in a local Intranet LIN. However, the method according to this embodiment provides additional advantages. FIG. 5 also explains a system 100 in accordance with yet another embodiment according to the second aspect of the present invention, i.e. a system for providing dose data from a cloud-based dose management system, CBDMS, in a local Intranet LIN.

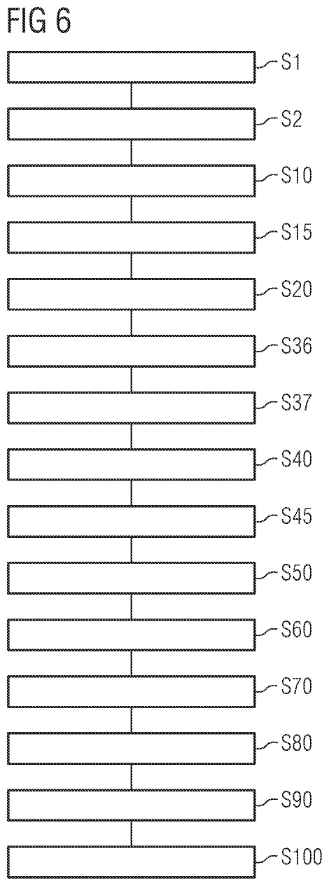

The flowchart in FIG. 6, which again shows schematic representations of individual method steps in association therewith, belongs to FIG. 5.

The present embodiment essentially describes the interaction of a local Intranet, for instance as described above with reference to one or all of FIGS. 1 to 4, with a national dose database or dose registry, abbreviated to national REG.

The method according to FIG. 5 and FIG. 6 is largely the same as or similar to the method according to FIGS. 5 and 6, so that only the differences therefrom are explained below, wherein these differences can be used and understood as alternatives, but can also be used and understood as possible supplements within the scope of the afore-described embodiments.

Accordingly, the case is described here in that the dose data provisioning module, DDBM, comprises dose registration module, DRM 16, instead of the proactive push module, PPM 14, wherein it is clear that a dose provisioning module, DDBM, can comprise both the dose registration module, DRM 16, and also the proactive push module, PPM, and also even the reactive pull module, RPM 12.

With reference to FIG. 5 and FIG. 6, the steps S10 to S20 are carried out with the optional steps S1 and/or S2, as described above.

Also in the present case, a trigger condition is checked by the dose data provisioning module, DDBM, namely the same trigger condition as was described above with respect to steps S36 and S37. The difference is that in the present embodiment the dose registration module, DRM 16, carries out the steps.

In step S60, the second dose data (with or without supplements) is finally provided to a registry site service system, REG 50, within the local Intranet LIN.

In a step S70, third dose data is then transmitted by the registry site service system, REG 50, based upon the second dose data DD2 to a national dose registry computing facility, NDRR 150. The third dose data DD3 can be identical to the second dose data DD2 (with or without supplements) or contain extracts therefrom, for instance, or comprise calculations based thereupon.

It is advantageous if the third dose data is mapped onto LOINC-compatible RadLex protocols (i.e. mapped correctly to one another), particularly if each individual scan of an exam is mapped onto a LOINC-compatible RadLex protocol.

In a step S80, the registry site service system, REG 50, receives dose benchmark data, DBD, from the national dose registry computing facility, NDRR 150.

In a step S90, the registry site service system, REG 50, then informs the dose registration module, DRM 16, of the availability of new dose benchmark data, DBD.

In a step S100, the dose registration module, DRM 16, uploads the new dose benchmark data, DBD, into the cloud-based dose management system, CBDMS 20. This new dose benchmark data is therefore in turn available inter alia as supplementary information relating to the second dose data DD2 or as part of the second dose data DD2, for instance for the reactive pull module, RPM 12 and/or for the proactive push module, PPM 14.

FIGS. 1 to 6 moreover illustrate in each case an embodiment of a system 100 according to embodiments of the present invention. As already mentioned repeatedly, these embodiments can also be combined so that a system 100 can also be provided, which has a reactive pull module, RPM 12, a proactive push module, PPM 14, and a dose registration module, DRM 16, or a selection thereof.

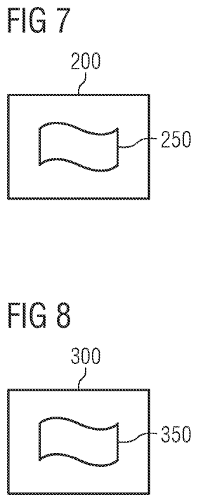

FIG. 7 shows a schematic block diagram to illustrate a computer program product 200 according to an embodiment of the third aspect of the present invention. The computer program product 200 comprises executable program code 250, which is embodied, when it is executed, to execute the method according to one of the embodiments of the first aspect of the invention, in particular the method as was explained based upon one or more of FIGS. 1 to 6.

FIG. 8 shows a schematic block diagram to illustrate a computer-readable, non-volatile data storage medium 300 according to an embodiment of the fourth aspect of the present invention. The one computer-readable, non-volatile data storage medium 300 comprises executable program code 350, which is embodied, when it is executed, to execute the method according to one of the embodiments of the first aspect of the invention, in particular the method, as was explained based upon one or more of FIGS. 1 to 6.

The computer-readable medium can also be e.g. a data memory such as a magnetic memory (e.g. magnetic core memory, magnetic tape, magnetic card, magnetic strip, magnetic bubble memory, drum module, hard disk, floppy disk or removable storage), an optical memory, e.g. a holographic memory, optical tape, Tesafilm, laser disk, Phasewriter (Phasewriter Dual, PD) or Ultra Density Optical (UDO)), a magneto-optical memory (e.g. miniDisc or magneto-optical disk (MO disk), a volatile semiconductor/solid body memory (e.g. random access memory (RAM), dynamic RAM (DRAM) or static RAM (SRAM)) or a non-volatile semiconductor/solid body memory (e.g. read only memory (ROM), programmable ROM (PROM), erasable PROM (EPROM), electrically EPROM (EEPROM), flash EEPROM (e.g. USB stick), ferro-electric RAM (FRAM), magnetoresistive RAM (MRAM) or phase-change RAM).

Although specific embodiments were illustrated and described here, it is apparent to the person skilled in the art that there are a plurality of alternatives and/or equivalent implementations. It should be recognized that the example embodiments or developments are only examples and are not intended to restrict the scope, the applicability or the configuration in any way. Rather, the abstract and detailed description above will supply adequate instructions to the person skilled in the art for implementing at least one preferred embodiment, wherein it is clear that different changes in function and arrangement of the elements, which are described in an example embodiment, do not extend from the application range shown in the appended claims and their legal equivalents. This application is generally intended to cover all adjustments or variations in the specific embodiments discussed here.

For instance, all processes, concepts, system components and/or method steps described in respect of dose data or dose data management can be applied similarly to contrast management or protocol management.

In the detailed description above, various features were combined in one or more examples in order to keep the disclosure brief. It is clear that the above description is to be illustrative and not restrictive. It should cover all alternatives, changes and equivalents which may be contained within the scope of the invention. Many other examples will become obvious to a person skilled in the art in the study of the above disclosure.

In order to enable a comprehensive understanding of the invention, a specific nomenclature of the invention is used which was used in the above disclosure. It is however apparent to a person skilled in the art in light of the specification contained therein that the specific details are not required to apply the invention. The above descriptions of special embodiments of the present invention are therefore shown for illustration and description purposes. They are not intended to be exhaustive or to restrict the invention to the precise embodiments disclosed above; obviously many modifications and variations are possible in respect of the afore-cited teaching. The embodiments were selected and described in order to best explain the fundamentals of the invention and its practical applications and therefore to provide other specialist personnel with the opportunity to best apply the invention and different embodiments with different modifications, as appears suitable for the respective use. In the entire specification, the terms “including” and “in which” are used as the equivalents of the respective terms “comprising” or “wherein”. Furthermore, the terms “first”, “second”, “third” etc. are only used as a reference and not intended to place numerical demands on the objects or to prespecify a specific sequence. In association with the present description and the claims, the connection “or” is to be understood as an inclusion (“and/or”) and not exclusively (“either . . . or”).

The example embodiments have been described for illustrative purposes. It will be obvious that the described features, steps and workflow may be varied in many ways. Such variations are not to be regarded as a departure from the scope of the present invention, and all such modifications as would be obvious to one skilled in the art are intended to be included within the scope of the following claims.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for providing dose data from a cloud-based dose management system in a local Intranet, comprising:
receiving, at a medicine data gateway within the local Intranet, study data according to at least one selection criterion from an image archiving and communication system, within the local Intranet;
uploading, via the medicine data gateway, at least first dose data based upon the study data received, into a cloud-based dose management system, the first dose data including or indicating at least one respective unique study-identifying characteristic value;
checking, via a dose data provisioning module executed in the medicine data gateway within the local Intranet, whether a trigger condition is present;
downloading via the dose data provisioning module, upon the checking indicating a presence of the trigger condition, second dose data based upon the first dose data from the cloud-based dose management system, the second dose data including or indicating a respective unique study-identifying characteristic value; and
providing the second dose data to a reporting system or another local apparatus within the local Intranet, either automatically or upon a provisioning condition being fulfilled.

2. The method of claim 1, wherein the unique study-identifying characteristic value is at least one of or is based on at least one of:
an accession number, representing a reference number in a radiology information system within the local Intranet;
a patient identification characteristic value; and
a StudyInstanceUID, designed to uniquely identify a study associated with a structured report.

3. The method of claim 1, wherein the dose data provisioning module includes a reactive pull module which provides a URL, callable by a local apparatus within the local Intranet, to request the second dose data via the local apparatus.
wherein upon the URL being called, the URL includes at least one respective unique study-identifying characteristic value;
wherein the trigger condition includes receiving of a call from the URL provided; and
wherein the second dose data is provided by the reactive pull module of the local apparatus.

4. The method of claim 3, wherein the reactive pull module provides the local apparatus with a service access token, checked by the reactive pull module within a scope of the URL call on the local device, to authenticate the call, and wherein the trigger condition includes a successful authentication of the service access token.

5. The method of claim 3, wherein the local apparatus is a radiology information system.

6. The method of claim 1, wherein the dose data provisioning module includes a proactive push module; and
wherein the trigger condition includes receiving of an item of information, from the cloud-based dose management system, indicating that a study was fully uploaded into the cloud-based dose management system.

7. The method of claim 1, wherein the medicine data gateway de-identifies the first dose data and uploads the first dose data in de-identified form into the cloud-based dose management system; and
wherein the medicine data gateway re-identifies the downloaded second dose data based upon the respective unique study-identifying characteristic value.

8. The method of claim 1, wherein information about at least one of a standard protocol mapping and standard reference values is provided as part of the second dose data or in addition to the second dose data.

9. The method of claim 1, wherein the provisioning condition is or includes a positive result of a check to determine whether an incomplete order is currently present on the reporting system, the incomplete order being linked to a unique study-identifying characteristic value, associated with the second dose data.

10. The method of claim 1, wherein the dose data provisioning module includes a dose registration module;
wherein upon a study being fully uploaded into the cloud-based dose management system, the cloud-based dose management system informs a dose registration module executed in the medicine data gateway;
whereupon the dose registration module provides the second dose data to a registry site service system as the local device within the local Intranet; and
wherein the registry site service system transmits third dose data, based upon the second dose data, to a national dose registry computing facility.

11. The method of claim 10, wherein the registry site service system receives dose benchmark data from the national dose registry computing facility, whereupon the registry site service system informs the dose registration module of availability of new dose benchmark data, whereupon the dose registration module uploads the new dose benchmark data into the cloud-based dose management system.

12. The method of claim 11, wherein the cloud-based dose management system additionally functions as at least one of a global dose registry, as a cloud-based contrast management system and as a cloud-based protocol management system.

13. A system for providing dose data from a cloud-based dose management system, comprising:
a medicine data gateway, within a local Intranet of the system, the medicine data gateway being embodied to execute a dose data provisioning module;
a reporting system, within the local Intranet; and
a cloud-based dose management system, outside of the local Intranet;
the system being configured to
receive, at a medicine data gateway within the local Intranet, study data according to at least one selection criterion from an image archiving and communication system, within the local Intranet;
upload, via the medicine data gateway, at least first dose data based upon the study data received, into a cloud-based dose management system, the first dose data including or indicating at least one respective unique study-identifying characteristic value;
check, via a dose data provisioning module executed in the medicine data gateway within the local Intranet, whether a trigger condition is present;
download via the dose data provisioning module, upon the checking indicating a presence of the trigger condition, second dose data based upon the first dose data from the cloud-based dose management system, the second dose data including or indicating a respective unique study-identifying characteristic value; and
provide the second dose data to a reporting system or another local apparatus within the local Intranet, either automatically or upon a provisioning condition being fulfilled.

14. A non-transitory computer program product storing executable program code, embodied, when executed, to execute the method of claim 1.

15. A non-transitory computer-readable, non-volatile data storage medium storing executable program code embodied, when executed, to execute the method of claim 1.

16. The method of claim 2, wherein the dose data provisioning module includes a reactive pull module which provides a URL, callable by a local apparatus within the local Intranet, to request the second dose data via the local apparatus.
wherein upon the URL being called, the URL includes at least one respective unique study-identifying characteristic value;
wherein the trigger condition includes receiving of a call from the URL provided; and
wherein the second dose data is provided by the reactive pull module of the local apparatus.

17. The method of claim 16, wherein the reactive pull module provides the local apparatus with a service access token, checked by the reactive pull module within a scope of the URL call on the local device, to authenticate the call, and wherein the trigger condition includes a successful authentication of the service access token.

18. The method of claim 4, wherein the local apparatus is a radiology information system.

19. The method of claim 2, wherein the dose data provisioning module includes a proactive push module; and
wherein the trigger condition includes receiving of an item of information, from the cloud-based dose management system, indicating that a study was fully uploaded into the cloud-based dose management system.

20. The method of claim 2, wherein the medicine data gateway de-identifies the first dose data and uploads the first dose data in de-identified form into the cloud-based dose management system; and
wherein the medicine data gateway re-identifies the downloaded second dose data based upon the respective unique study-identifying characteristic value.
